(19) United States
(12) Patent Application Publication
(10) Pub. No.: US 2019/0274653 A1
(43) Pub. Date: Sep. 12, 2019

(54) METHOD AND APPARATUS FOR ARTIFACT REDUCTION FOR JOINT REGION IN STEP AND SHOOT COMPUTED TOMOGRAPHY

(71) Applicant: FMI Medical Systems Co., Ltd., Zhejiang (CN)

(72) Inventors: Hongbin Guo, Champaign, IL (US); Chuang Miao, Macedonia, OH (US); Abdelaziz Ikhlef, Hudson, OH (US); Xuelin Cui, Solon, OH (US)

(73) Assignee: FMI Medical Systems Co., Ltd.

(21) Appl. No.: 15/913,540

(22) Filed: Mar. 6, 2018

(51) Int. Cl. A61B 6/00; G06T 11/00; G06T 3/40; G06T 7/00

(52) U.S. Cl. CPC A61B 6/5282; G06T 11/005; G06T 2207/10081; G06T 7/0012; G06T 7/97; G06T 3/4007

(57) ABSTRACT

A computed tomography (CT) system includes a rotatable gantry having an opening to receive an object to be scanned, an x-ray tube having an anode, the x-ray tube positioned on the gantry to generate x-rays from a focal spot of the anode and through the opening, a pixelated detector positioned on the gantry to receive the x-rays from which CT projection data is generated, and a computer. The computer programmed to acquire step-and-shoot (SAS) full scan CT projection data for a first scan and for a first rotation, and for a second scan and for a second rotation, wherein the first scan is axially offset from the second scan, interpolate across the first and second scans to generate interpolated projection data, and reconstruct an image based on the interpolated projection data.

### TECHNICAL FIELD

This disclosure relates generally to diagnostic imaging and, more particularly, to an apparatus and method of reducing artifacts for a joint region in a step and shoot computed tomography (CT) system.

### BACKGROUND

Typically, in computed tomography (CT) imaging systems, a rotatable gantry includes an x-ray tube, detector, data acquisition system (DAS), and other components that rotate about a patient that is positioned at the approximate rotational center of the gantry. X-rays emit from the x-ray tube, are attenuated by the patient, and are received at the detector. The detector typically includes a photodiode-scintillator array of pixelated elements that convert the attenuated x-rays into photons within the scintillator, and then to electrical signals within the photodiode. The electrical signals are digitized and then received within the DAS, processed, and the processed signals are transmitted via a slipring (from the rotational side to the stationary side) to a computer or data processor for image reconstruction, where an image is formed.

The gantry typically includes a pre-patient collimator that defines or shapes the x-ray beam emitted from the x-ray tube. X-rays passing through the patient can cause x-ray scatter to occur, which can cause image artifacts. Thus, x-ray detectors typically include an anti-scatter grid (ASG) for collimating x-rays received at the detector. Imaging data may be obtained using x-rays that are generated at a single polychromatic energy. However, some systems may obtain multi-energy images that provide additional information for generating images.

Third generation multi-slices CT scanners typically include a detector assembly having scintillator/photodiodes arrays positioned in an arc, where the focal spot is the center of the corresponding circle. The material used in these detectors generally has scintillation crystal/photodiode arrays, where the scintillation crystal absorbs x-rays and converts the absorbed energy into visible light. A photodiode is used to convert the light to an electric current. The reading is typically proportional and linear to the total energy absorbed in the scintillator.

X-ray Tomography is widely used in clinical disease diagnosis. However, a fundamental circular cone beam (CCB) scan suffers from cone beam artifacts due to data incompletion, (i.e. projection data does not satisfy a data sufficiency condition (DSC)). That is, CCB data fundamentally includes insufficient data and there is thus no stable reconstruction. In CT scanners, FDK-type (Feldkamp-Davis-Kress) algorithms are the most commonly used analytic reconstruction method. FDK-type methods are extended from two-dimensional reconstruction methods which include 180 degrees plus fan angle projection data to approximate exact reconstruction in fan beam geometry. Unfortunately, some regions at the ends of the volume do not satisfy this condition even for a full circular, 360-degree scan, and the largest region that can be reconstructed contains the voxels having 180 degrees plus fan angle projection data. If a reconstruction volume z-coverage is set to the full illumination range at z-axis, some regions at the end slices of the volume do not satisfy this condition either even for a full circular, 360-degree, scan, which can lead to image artifacts.

Methods have been developed to reduce image artifacts in CCB scans. According to one known method, reconstruction z-coverage is shortened and only a partial data set is used. However, true z-coverage in this method is less than that of the system, and partial radiation dose may be wasted.

According to another known method for step and shoot CCB scanning, cone beam artifacts are reduced by overlap scanning and individually reconstructing image volumes from two consecutive scans. After image reconstruction of each scan, the joint overlap slices are combined, and weights are quantified by the amount of extrapolation used in the reconstruction. However, this computationally costly method may alter noise properties in reconstructed images, and inaccurately extrapolated data may be used to reconstruct the end slices of each scan. This can result in large errors in the final images, and performance is largely dependent on the weighing strategy, which may include an estimation of the amount of extrapolation used in the reconstruction.

Thus, there is a need to improve CCB scans.

### BRIEF DESCRIPTION

The disclosure is directed toward an apparatus, method of fabricating, and method of using a reference detector in computed tomography (CT)

A computed tomography (CT) system includes a rotatable gantry having an opening to receive an object to be scanned, an x-ray tube having an anode, the x-ray tube positioned on the gantry to generate x-rays from a focal spot of the anode and through the opening, a pixelated detector positioned on the gantry to receive the x-rays from which CT projection data is generated, and a computer. The computer programmed to acquire step-and-shoot (SAS) full scan CT projection data for a first scan and for a first rotation, and for a second scan and for a second rotation, wherein the first scan is axially offset from the second scan, interpolate across the first and second scans to generate interpolated projection data, and reconstruct an image based on the interpolated projection data.

A method of imaging includes passing an object through an opening of a rotatable gantry to be scanned, receiving x-rays that pass through the object, in a pixelated detector positioned on the gantry, from which CT projection data is generated, acquiring step-and-shoot (SAS) full scan CT projection data for a first scan and for a first rotation, and for a second scan and for a second rotation, wherein the first scan is axially offset from the second scan, interpolating across the first and second scans to generate interpolated projection data, and reconstructing an image based on the interpolated projection data.

A computer readable storage medium having stored thereon a computer comprising instructions, which, when executed by a computer, cause the computer to acquire step-and-shoot (SAS) full scan CT projection data for a first scan and for a first rotation of a gantry, and for a second scan and for a second rotation of the gantry, wherein the first scan is axially offset from the second scan, interpolate across the first and second scans to generate interpolated projection data, and reconstruct an image based on the interpolated projection data.

Various other features and advantages will be made apparent from the following detailed description and the drawings.

### BRIEF DESCRIPTION OF THE DRAWINGS

FIG. 1 is a perspective view of a CT imaging system.

FIG. 2 is a planar cross-section of the system illustrated in FIG. 1.

FIG. 3 is an example of an imaging chain.

FIG. 4 is an example of a detector module.

FIG. 5 illustrates data incompletion in Radon space.

FIG. 6 shows, in one example, 4 slices at each end of a volume that do not have enough projections to reconstruct a full slice with FDK.

FIG. 7 shows consecutive step-and-shoot scans having a space between consecutive scans and showing a pixel having interpolation performed thereon, according to the disclosure.

FIG. 8 represents an exemplary parallel projection map of one SAS view at a plane passing a Z-axis.

FIG. 9 illustrates an algorithm according to the disclosure.

### DETAILED DESCRIPTION

The operating environment of disclosed examples is described with respect to a multislice computed tomography (CT) system. Examples are described with respect to a “third generation” CT scanner, however it is contemplated that the disclosed examples are applicable to other imaging systems as well, and for CT systems having more or less than the illustrated sixty-four-slice system.

The present disclosure includes a method to substitute current reference channels used for projection data normalization by a factor based on a feedback current (mA) generated from a high voltage generator.

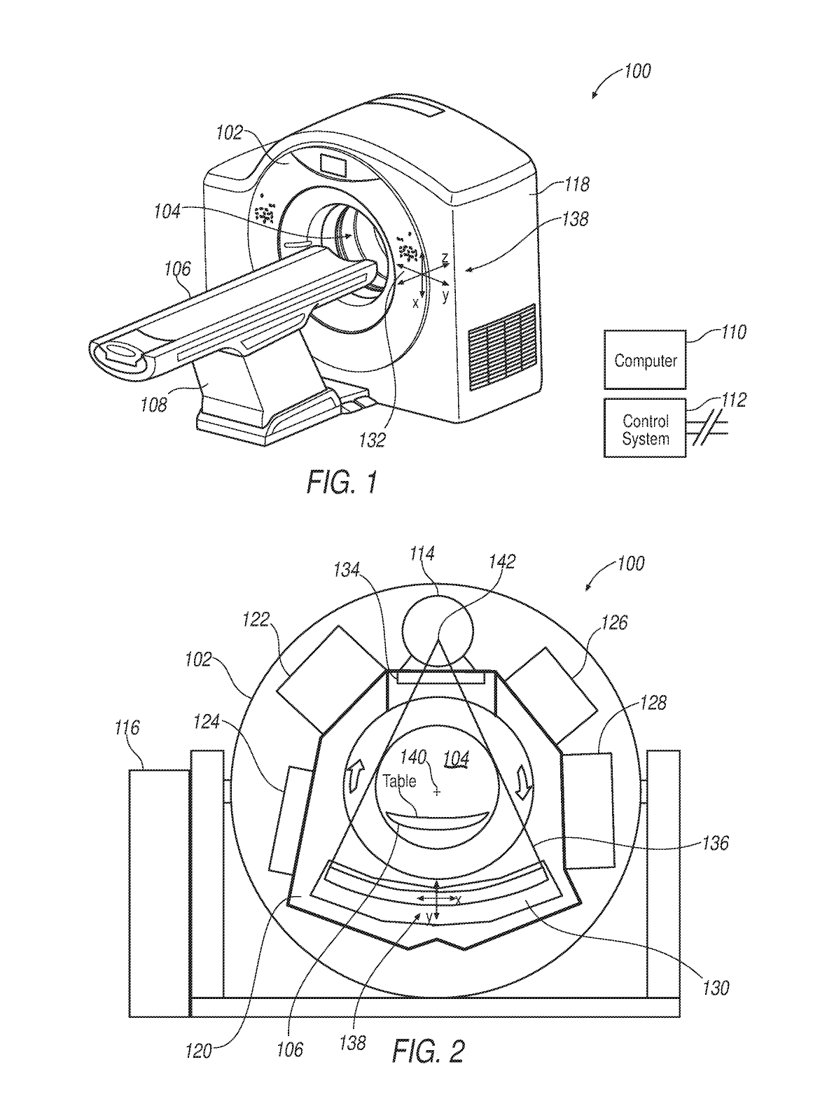

Referring to FIGS. 1 and 2, a computed tomography (CT) system 100 includes a gantry 102 having an opening 104. A patient table 106 is positioned on a support structure 108, and patient table 106 is axially controllable such that a patient (not shown) positioned on table 106 may be positioned within opening 104. A computer system 110 provides operator instructions and other control instructions to a control system 112. Computer system 110 also may include image reconstruction programs, or an image reconstructor may be provided as a separate processing unit. Control system 112 provides control commands for operating gantry 102, an x-ray tube 114, and a gantry motor controller 116, as examples. Gantry 102 includes a cover or enclosure 118, which provides for aesthetic improvement, safety, etc.

Gantry 102 includes a rotatable base 120, on which is mounted x-ray tube 114, a heat exchanger 122, a data acquisition system (DAS) 124, an inverter 126, a high-voltage generator 128 for generating high voltage in x-ray tube 114, and a detector assembly 130, as examples. System 100 is operated with commands entered by a user into computer 110. Gantry 102 may include gantry controls 132 located thereon, for convenient user operation of some of the commands for system 100. Detector assembly 130 includes a plurality of detector modules (not shown), which include an anti-scatter grid (ASG), scintillators, photodiodes, and the like, which detect x-rays and convert the x-rays to electrical signals, from which imaging data is generated. Gantry 102 includes a pre-patient collimator 134 that is positioned to define or shape an x-ray beam 136 emitted from x-ray tube 114. Although not shown, a shape filter may be positioned for instance between x-ray tube 114 and pre-patient collimator 134.

In operation, rotatable base 120 is rotating about the patient, and table 106 is enabled to move the patient axially into the opening 104. When a desired imaging location of the patient is proximate an axial location where x-ray beam 136 will be caused to emit, x-ray tube 114 is energized and x-ray beam 136 is generated from a focal spot within x-ray tube 114. The detectors receive x-rays, some of which have passed through the patient, yielding analog electrical signals are digitized and passed to DAS 124, and then to computer 110 where the data is further processed to generate an image. The imaging data are stored on computer system 100 and images may be viewed. An X-Y-Z triad 138, corresponding to a local reference frame for components that rotate on rotatable base 120, defines a local directional coordinate system in a gantry circumferential direction X, a gantry radial direction Y, and gantry axial direction Z. Accordingly, and referring to triad 138, the patient passes parallel to the Z-axis, the x-rays pass along the Y axis, and the rotational components (such as detector assembly 130) rotate in a circumferential direction and in the X direction, and about an isocenter 140 (which is a centerpoint about which rotatable base rotates, and is an approximate position of the patient for imaging purposes). A focal spot 142 is illustrated within x-ray tube 114, which corresponds to a spot from which x-ray beam 136 emits.

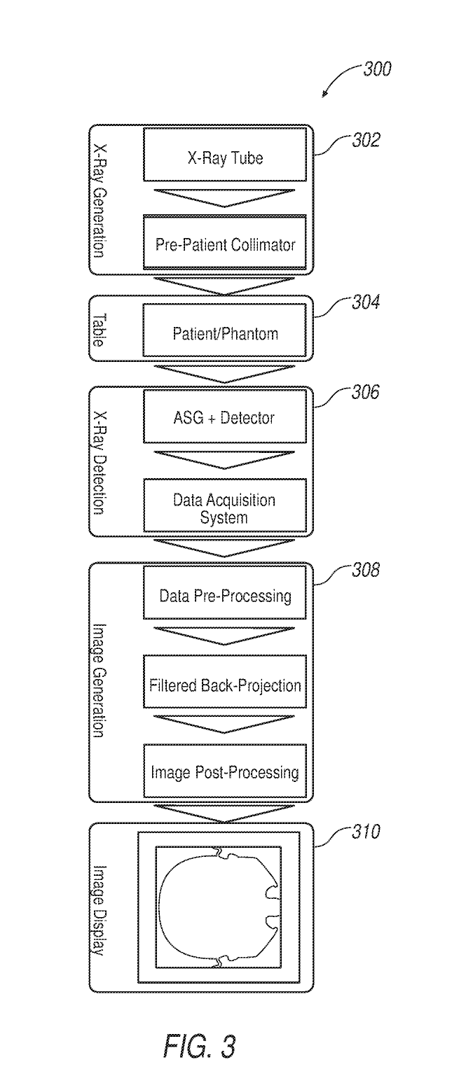

FIG. 3 illustrates an exemplary image chain 300, consistent with the operation described with respect to FIGS. 1 and 2. X-ray generation 302 occurs, using x-ray tube 114 and passing x-rays through pre-patient collimator 134, during which patient table 106 passes 304 through opening 104 of gantry 102. In one example table 106 may have a patient thereon, and in another example a phantom may be used for calibration purposes.

X-ray detection 306 occurs when x-rays having been emitted from x-ray tube 114 pass to detector assembly 130. An anti-scatter grid (ASG) prevents x-ray scatter (emitting for example from the patient as secondary x-rays and in a direction that is oblique to x-ray beam 136), by generally filtering x-rays that emit from x-ray tube 114. DAS 124 processes signals received from detector assembly 130. Image generation 308 occurs after the digitized signals are passed from a rotating side of gantry 102 (on rotatable base 120) to a stationary side, via for instance a slip-ring.

Image generation 308 occurs in computer system 110, or in a separate processing module that is in communication with computer system 110. The data is pre-processed, and image views or projections are used to reconstruct images using known techniques such as a filtered backprojection (FBP). Image post-processing also occurs, after which the images may be displayed 310, or otherwise made available for display elsewhere (such as in a remote computing device).

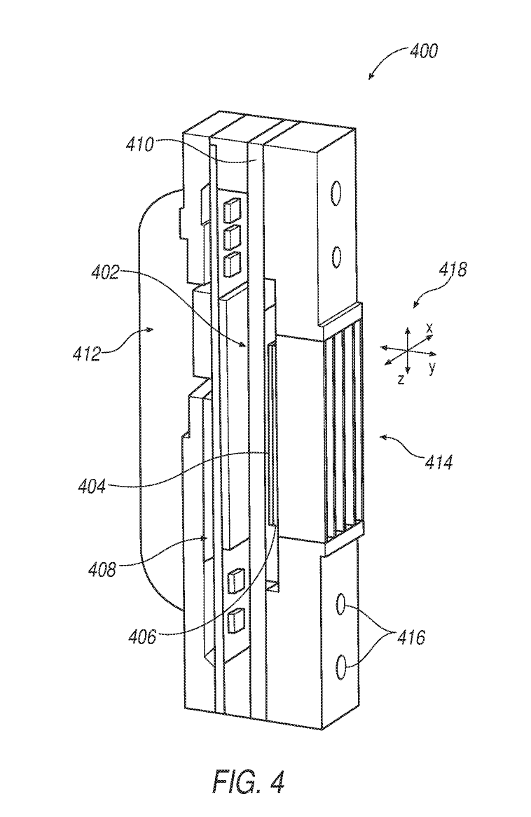

FIG. 4 illustrates an exemplary detector module 400 that is one of a plurality of modules for use in detector assembly 130. A diode-scintillator array 402 includes a pixelated scintillator 406 positioned on a pixelated photodiode array 404. The photodiode array 404 may be either a front-lit or a back-lit type of photodiode. The diode-scintillator array 402 is positioned on an A/D board 408 that includes electronics components for signal processing, wherein analog electrical signals from diode-scintillator array 402 are digitized and then passed to DAS 124. Diode-scintillator array 402 is positioned on a base substrate 410 that may include a ceramic or other solid base material. A heat sink 412 is in thermal contact with A/D board 408 for providing enhanced cooling to the electronics located on A/D board 408. Detector module 400 also includes an anti-scatter grid (ASG) 414 that, in one embodiment, includes a plurality of plates (a few exemplary plates are shown) that are approximately parallel with a Y-Z plane of detector assembly 130. ASG 414, in the illustrated example, includes mount holes 416 which may be used for mounting module 400 to detector assembly 130 and aligning it therewith. FIG. 4 illustrates a triad 418 that illustrates corresponding X-Y-Z coordinates, as illustrated also in FIG. 1.

Purposes of the disclosure are to reduce cone beam artifacts, improve image quality, and improve dose efficiency for CT x-ray systems. The advantages include a volume of a full scan coverage in Z that may be reconstructed. Cone beam artifacts are suppressed significantly. Dose utilization efficiency is improved. Data extrapolation is not needed to rearrange the original data that will introduce extrapolation errors. The disclosed algorithm is computational efficient without increasing computational cost.

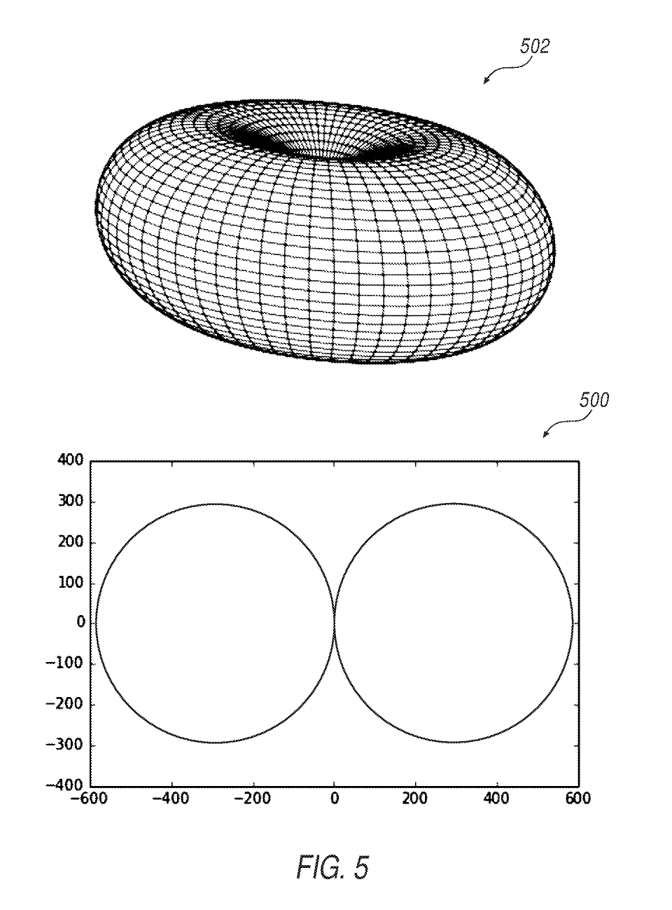

FIG. 5 illustrates a data incompletion in Radon space, represented in both a 2-dimensional (XZ) illustration 500 (the filled regions having projection data) and a 3-dimensional illustration 502. Thus, in FIG. 5, radon space of a CCB scan shows data incompletion.

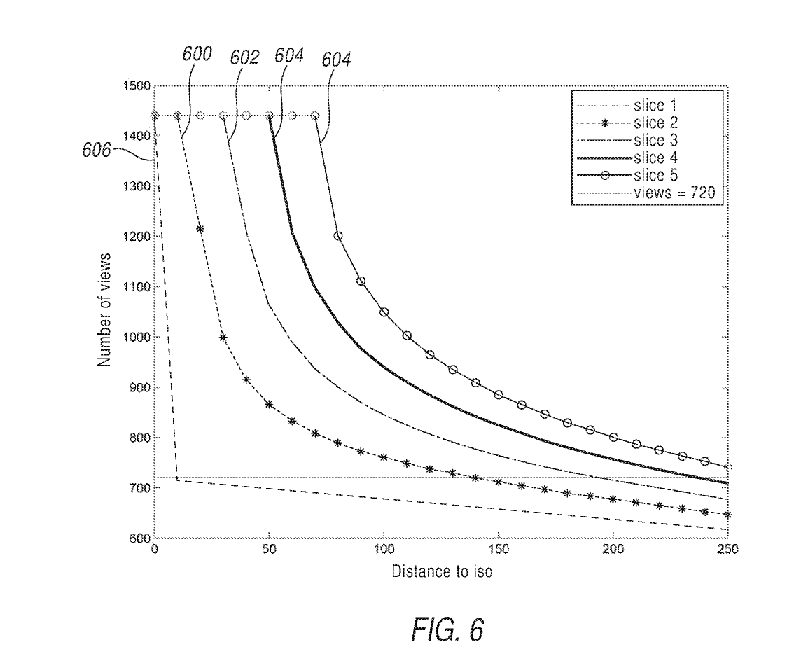

FIG. 6 shows, in one example, that there are 4 slices 600, 602, 604, 606 at each end of a volume that do not have enough projections to reconstruct a full slice with FDK, if the FOV is set to 500 mm, where a geometry of a 64-row scanner is used. In computed tomography (CT) scanners, FDK-type (Feldkamp-Davis-Kress) algorithms are some of the more commonly used analytic reconstruction methods. For FDK-type reconstruction the largest reconstruction region typically contains voxels having projections with angle span more than 180 degrees plus fan angle projection data. The two end slices only have one center voxel that satisfies the FDK reconstruction condition. If extrapolation is applied to the unknown angles, strong artifacts may be introduced. Due to cone angle effects the region with 180 degrees plus fan angle projections may have artifacts. Thus, the reconstruction volume is usually set to less than the illumination range at Z-axis.

That is, the number of illuminated views versus voxel distances to the Z-axis for the first 5 slices are shown. In the computation, the geometry of a 64-row scanner is used and the reconstruction volume z-coverage is set to the full illumination range at the Z-axis (i.e. 40 mm). Slices 1 to 4 (elements 600 to 604) do not have enough projections to reconstruct the full slice with FDK, if the FOV is set to 500 mm, in this example. That is, slice 606 only has the one center voxel that satisfies the FDK reconstruction condition.

As such, in summary and according to the disclosure, in one form an x-ray CT reconstruction strategy is to reduce cone beam artifacts and improve dose efficiency. A method is disclosed to reduce cone beam artifacts in end slices using complementary projection data from consecutive scans. The cone beam artifacts in end slices are caused by incomplete data in radon space, as can be seen in FIG. 5. By utilizing the geometric symmetry of CT scanner, missing data in one scan can be compensated using data collected from consecutive scans. Thus, the disclosure has at least the following improvements:

As indicated, regions at the ends of the volume, shown in FIG. 6, do not satisfy the data requirement for reconstruction of the voxels even with a full circular, 360-degree, scan as shown in FIG. 5. The largest region that can be reconstructed contains the voxels having 180-degree plus fan angle projection data. Thus, in this example, if the reconstruction volume Z-coverage is set to the full illumination range at the z-axis, some regions at the end slices of the volume do not satisfy the data requirement for reconstruction of the voxels even with a full circular, 360-degree, scan.

For a FDK type reconstruction the largest reconstruction region contains the voxels which have projections with an angle span that is more than 180 degrees plus the fan angle projection data. FIG. 6 shows that there are 4 slices at each end of the volume do not have enough projections to reconstruct the full slice with FDK if we set the FOV to 500 mm, where the geometry of a 64-row scanner is used. The two end slices only have the ONE center voxel satisfy FDK reconstruction condition. If apply extrapolation to the unknown angles strong artifacts may be introduced. Due to cone angle effects even the region with 180 degrees plus fan angle projections may have artifacts. Thus, the reconstruction volume is usually set to less than the illumination range at z-axis.

1) Full scan coverage reconstruction in the Z-direction with reduced artifacts. Previous methods reconstruct smaller coverage due to dropping scan data to address cone artifacts at the end slices.

2) A novel method is disclosed to utilize complementary data from consecutive scans to reduce cone beam artifacts.

3) Dose efficiency is improved.

4) Extrapolation is not necessary to rearrange the original data, which can introduce extrapolation errors.

According to the disclosure, sequential CCB scans, also called step-and-shoot (SAS) scanning, are used. In such scans, although there is not complete data for an exactly reconstruction, neighboring circular scans provide compensation data, according to the disclosure, to each other and within a region between two circular trajectories. Thus, improved images can be obtained for a central region with FDK-type methods by employing data from consecutive scans.

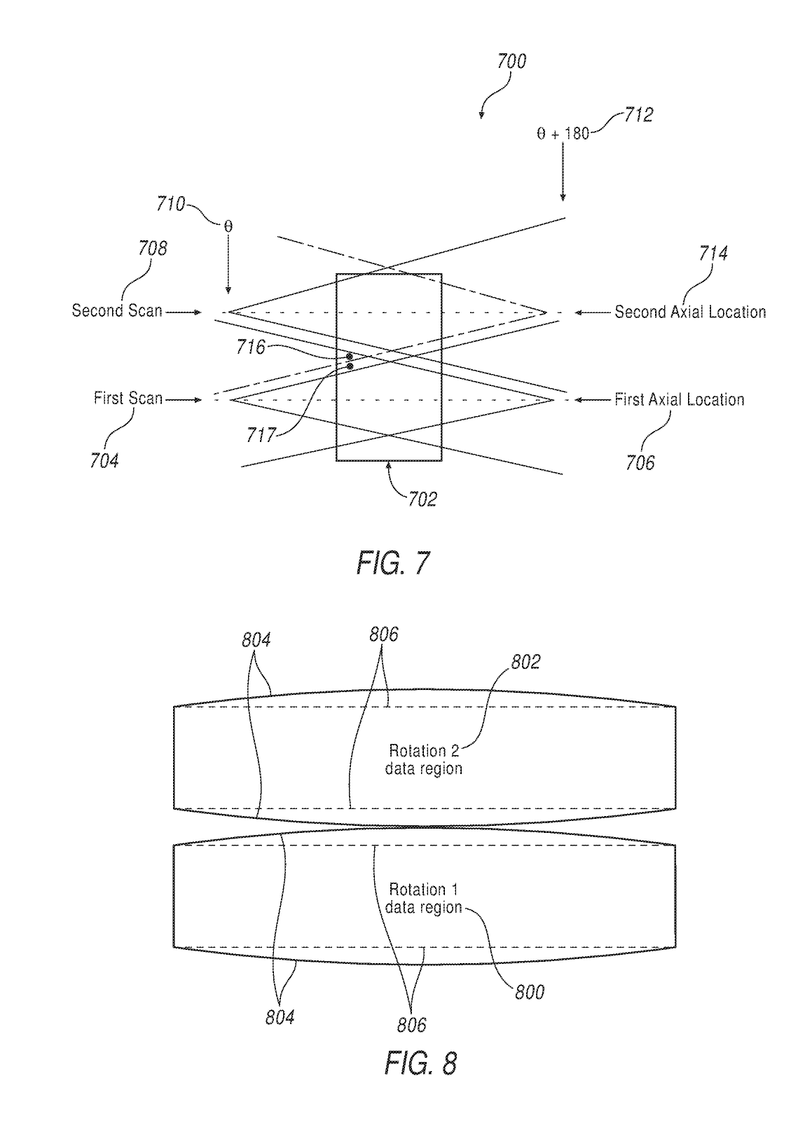

FIG. 7 illustrates SAS data acquisition, according to the disclosure. Referring to FIG. 7, a scenario 700 shows an object 702 to be scanned in two sequential SAS scans. A first scan 704 is performed at a first axial location 706 for a full 360-degree scan. Scenario 700 illustrates a 2-dimensional view of first scan 704, showing projection data obtained from an angle θ (710) and from an angle θ+180° (712). A second scan 708 is performed at a second axial location 714 that is axially offset from first axial location 706. An illustrative location or voxel 716 is located within object 702, and as can be seen, is captured outside of first and second scans 704, 708, when viewed from angle θ (710) for first scan 704, and when viewed from angle θ+180° (712) for second scan 714. Accordingly, first scenario 700 represents an exemplary voxel 716, whose data may be interpolated according to the disclosure.

Referring to FIG. 8, the regions described above with respect to FIG. 7 are shown therein. That is, a first rotation 800 and a second rotation 802 are shown. FIG. 8 thereby represents an exemplary parallel projection map of one SAS view at a plane passing a Z-axis. Solid curves bounded by 804, for each rotation, enclose illuminated regions. Voxels at the Z-axis are fully illuminated, while there is a gap between two rotations for off-center regions.

The fundamental FDK algorithm is extended from a two-dimensional reconstruction algorithm. To mitigate cone beam artifacts, different weight strategies are added to the basic FDK. In practice the fan geometry is usually re-binned to a parallel geometry. The use of re-binning and weights for the FDK algorithm is shown in Algorithm 1.

Algorithm 1. Weighted re-binning FDK algorithm

Step 1: Starting with the circular scanning data of a full scan, 360
degrees, with multi-row cylindrical detector panel p(γ,v,β) Here γ
and β represent fan angles of each channel and source position
angle of each projection, respectively.
Step 2: Re-binning the projection data p(γ,v,β) from fan geometry
to parallel geometry for each row v to produce p(υ,v,θ). Here
(υ,v) is a local coordinate for a virtual detector plane passing iso-
center of the CT system.
Step 3: Filtering the re-binned projection by kernel h(υ).
Step 4: Applying cosine weight.
Step 5: Performing weighted back-projection.

Mathematically this can be expressed as follows:

f(x)=1/2∫₀^{2π} cos(v)W(v)∫_{-∞}^{∞}p(u′,v,θ)h(u−u′)du′dθ EQN. 1,

Here u and v are actually functions: u=u(x,θ) and v=v(x,θ).

If a volume is reconstructed for the full Z-coverage at the iso-center axis, the end images reconstructed by Algorithm 1 will have artifacts since some voxels does not have 180 degrees parallel projections, which is the basic requirements for FDK reconstruction, and application-dependent information compensation may relieve this issue to some extent.

For purposes of this disclosure, the interest is in the middle region of the image for SAS scans. At a joint region between two consecutive circular scans, as shown, two rotations provide partial/complete complementary information each other at joint places. For example, the rays passing along the iso-center axis have complete complementary as shown in FIG. 7. The exemplary point 716, is located in the sub-volume of first scans, 704, however rotation does not pass the location at this view while the second rotations 708 at the opposite views can provide a projection passing this point. It might seem that for the joint region between two consecutive rotations there is sufficient 360-degree data everywhere. Unfortunately, this is not the case. For rays away from the iso-center axis there exists a gap between the projection data from two rotations, as seen in FIG. 7 at location 717. That is, as seen in FIG. 8, a cross-section of the volume is shown at one parallel view. Solid curves 804 enclose regions that are the illuminated region. Thus, voxels at the iso-center axis are all illuminated while there is a gap between two rotations for off-center positions. It is noted that the size of the gap is exaggerated, for illustration purposes, instead of proportional to an actual size.

Referring to FIG. 8, known systems merely use the data enclosed inside the dashed black rectangles 806, and data outside rectangles 806 is typically dropped. This strategy implies to shorten the step size of patient table movement in practical clinical implementation. However, doing so thereby increases dose to the patient, to obtain sufficient data.

Thus, according to the disclosure and to reduce the dose to patients, an interpolation is performed to fill in the gap between the two rotations. The disclosed algorithm is present in Algorithm 2, along with the following and corresponding discussion.

Remark 1: During the re-binning step, the parallel projection data of different circular trajectories are aligned to have the same projection angles. This is done so that the back-projection step can accurately locate the opposite projection from the neighboring rotation when it is needed.

Remark 2: The patient table movement step size must be sufficiently accurate, within certain tolerance, so that the assumed geometry in the back-projection is consistent with the merged data of consecutive rotations.

Because the interpolation is used to estimate the gap between two rotations in the disclosed method, the accuracy has been verified by simulated data and scanned physical phantom data. That is, all experimental data are obtained with the geometry of a 64-row scanner, having 64 row detectors each and having a height of 0.625 mm along the Z-axis, thus the Z-coverage at the Z-axis is 40 mm, and 840 channels at the X-axis direction with an arc angle 54.4 degrees. The exemplary scanner has a maximum FOV of 500 mm, and a cone angle of approximately 2 degrees. Exemplary data was obtained with the same protocol: SAS, 120 kVp, 200 mA and 1440 projections per rotation at a speed 1 second per rotation. Two rotations were simulated or performed for both experiments.

Experiment 1: A simulated FORBILD head phantom was projected and then reconstructed with a FOV of 350 mm. This phantom, having a complicated high contrast structure, presents a challenge object and thus it is good for reconstruction performance evaluation. The total number of slices of the reconstructed images is 128 slices with separation 0.625 mm. The end slice of the first rotation, i.e. 64th slice, has high resolution, and not having distortion, black shading, or other artifacts caused by data missing or extrapolation related inaccuracy.

Experiment 2: Abdomen section of human body phantom was scanned with 80 mm shift from iso-center and the body section is reconstructed with FOV 512 mm with 80 mm center shift correspondingly. This experiment demonstrates the feasibility of interpolation between two rotations for regions far from the Z-axis in end slices where the gap has the maximum extent. Again, the total number of slices of the reconstructed images is 128 slices with a separation of 0.625 mm. When the body center has 80 mm offset from the Z-axis, the regions of the abdomen section is more than 200 mm away from z-axis. Thus, there exists a large data gap between two rotations. A corresponding image was obtained, having no distortion, black shading, or other strong artifacts caused by data missing or extrapolation related inaccuracy.

Algorithm 2. SAS reconstruction by weighted re-binning FDK algorithm
from complementary projection data.

```
Step 1: Re-binning the projection data p(γ,v,β) from fan geometry
to parallel geometry for each row ν to produce p(u,v,θ).
Step 2: Filtering the re-binned projection by kernel h(u).
Step 3: Applying cosine weight to obtain q(u,v,θ).
Step 4: Performing weighted back-projection to reconstruct image
volume from complementary projection data. Pseudo-code is listed
below:
    1) For every voxel x,
    2) For views θₖ ∈ [0, π), k = 1,2...,K
        • For half turns m = 0, 1.
        • If x is illuminated by some Axial scan step at this
          view θₖ, do regular interpolation within this Axial
          rotation and compute the weight, else use
          complementary data, θₖ + π from next or previous
          rotation, to do interpolation across neighboring two
          scans and determine the weight.
        • End for half turns.
    3) Normalize weights over half turns.
    4) f (x) = f (x) + Σₘ₌₀¹ w(m, k, x) * q(u, v, m, k).
    5) End for views.
    6) End for voxel x.
```

Thus, disclosed is an algorithm for circular axial CT reconstruction. The disclosed algorithm does not discard any projection data. As such, it is dose efficient and it can reconstruct a larger volume than existing algorithms. In the disclosed algorithm the projection data of consecutive rotations are joined as a whole to complement each other. The large sampling gap between to rotations are filled with interpolation by using the data across two rotations. The algorithm has been validated with a simulated data and a physical phantom scan data. With the large cone angle data, no matter the high contrast case or off-center region, the reconstructed images do not show strong artifacts at joint slices.

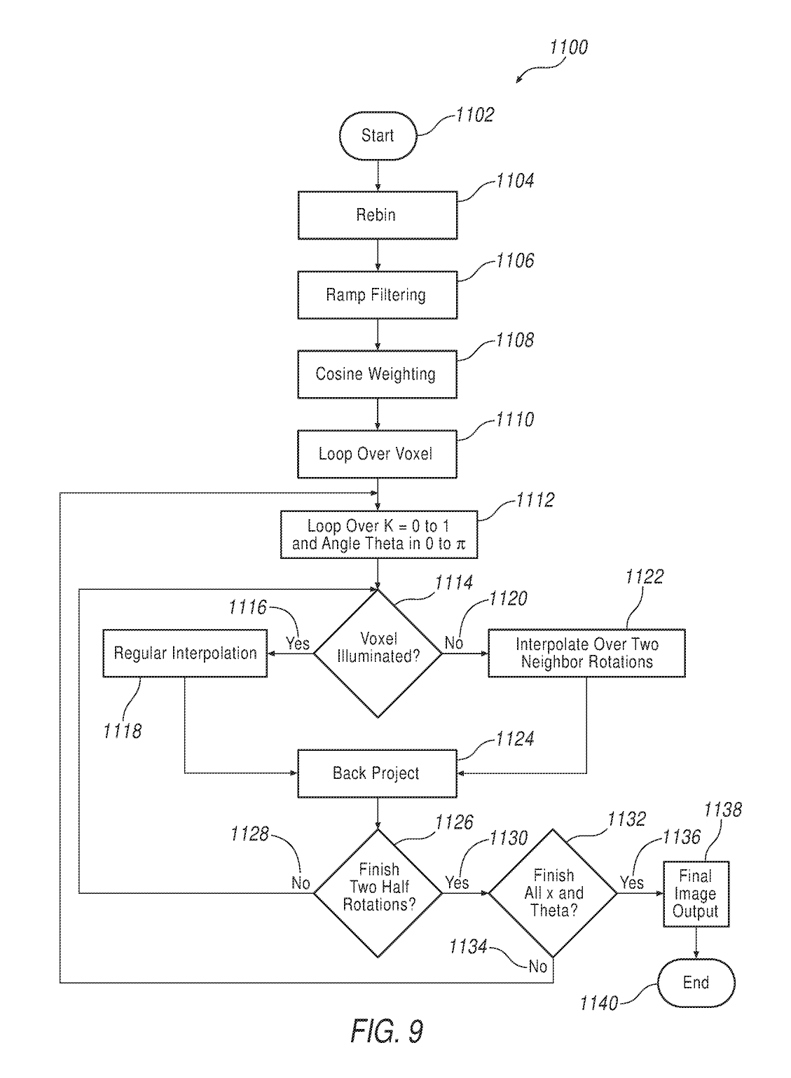

The overall algorithm 1100 is shown in FIG. 9. Starting at step 1102, acquired SAS imaging data is over sequential image volumes. Data is re-binned at step 1104, and ramp filtering is applied at step 1106. Cosine weighting is applied at step 1108, and the first voxel is considered at step 1110, with all voxels looped over K, at step 1112, from 0 to 1, and angle θ from zero to π. At step, next is considered whether the voxel is illuminated at step 1114. If so 1116, then a regular or known interpolation is performed at step 1118. However, if the voxel is not illuminated 1120 (such as falling between scans as described above with respect to FIG. 7), then data is interpolated over two neighbor rotations at step 1122. At step 1124, control moves to the step of back-projecting. At step 1126 is considered whether two half-rotations have been finished. If not 1128, then control moves back to step 1114 to consider the next voxel. If so 1130, then next is to consider all X and θ at step 1132. If all X and θ have not been finished 1134, then control moves back to step 1112 to continue the loop overall K from 0 to 1, and angle θ from zero to π. Once all X and θ have been finished 1136, then a final image is output at step 1138 and the process ends at step 1140.

Thus, according to the disclosure, a computed tomography (CT) system includes a rotatable gantry having an opening to receive an object to be scanned, an x-ray tube having an anode, the x-ray tube positioned on the gantry to generate x-rays from a focal spot of the anode and through the opening, a pixelated detector positioned on the gantry to receive the x-rays from which CT projection data is generated, and a computer. The computer programmed to acquire step-and-shoot (SAS) full scan CT projection data for a first scan and for a first rotation, and for a second scan and for a second rotation, wherein the first scan is axially offset from the second scan, interpolate across the first and second scans to generate interpolated projection data, and reconstruct an image based on the interpolated projection data.

Also according to the disclosure, a method of imaging includes passing an object through an opening of a rotatable gantry to be scanned, receiving x-rays that pass through the object, in a pixelated detector positioned on the gantry, from which CT projection data is generated, acquiring step-and-shoot (SAS) full scan CT projection data for a first scan and for a first rotation, and for a second scan and for a second rotation, wherein the first scan is axially offset from the second scan, interpolating across the first and second scans to generate interpolated projection data, and reconstructing an image based on the interpolated projection data.

And, according to the disclosure, a computer readable storage medium having stored thereon a computer comprising instructions, which, when executed by a computer, cause the computer to acquire step-and-shoot (SAS) full scan CT projection data for a first scan and for a first rotation of a gantry, and for a second scan and for a second rotation of the gantry, wherein the first scan is axially offset from the second scan, interpolate across the first and second scans to generate interpolated projection data, and reconstruct an image based on the interpolated projection data.

When introducing elements of various embodiments of the disclosed materials, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While the preceding discussion is generally provided in the context of medical imaging, it should be appreciated that the present techniques are not limited to such medical contexts. The provision of examples and explanations in such a medical context is to facilitate explanation by providing instances of implementations and applications. The disclosed approaches may also be utilized in other contexts, such as the non-destructive inspection of manufactured parts or goods (i.e., quality control or quality review applications), and/or the non-invasive inspection or imaging techniques.

While the disclosed materials have been described in detail in connection with only a limited number of embodiments, it should be readily understood that the embodiments are not limited to such disclosed embodiments. Rather, that disclosed can be modified to incorporate any number of variations, alterations, substitutions or equivalent arrangements not heretofore described, but which are commensurate with the spirit and scope of the disclosed materials. Additionally, while various embodiments have been described, it is to be understood that disclosed aspects may include only some of the described embodiments. Accordingly, that disclosed is not to be seen as limited by the foregoing description, but is only limited by the scope of the appended claims.

What is claimed is:

1. A computed tomography (CT) system, comprising:
a rotatable gantry having an opening to receive an object to be scanned;
an x-ray tube having an anode, the x-ray tube positioned on the gantry to generate x-rays from a focal spot of the anode and through the opening;
a pixelated detector positioned on the gantry to receive the x-rays from which CT projection data is generated; and
a computer programmed to:
acquire step-and-shoot (SAS) full scan CT projection data for a first scan and for a first rotation, and for a second scan and for a second rotation, wherein the first scan is axially offset from the second scan;
interpolate across the first and second scans to generate interpolated projection data; and
reconstruct an image based on the interpolated projection data.

2. The CT system of claim 1, wherein the computer is programmed to:
identify a voxel that is not illuminated in at least a portion of the first scan and at a given angle theta; and
generate the interpolated projection data based on the identified voxel.

3. The CT system of claim 2, wherein the computer is further programmed to generate the interpolated projection data using the second scan and based on the given angle theta.

4. The CT system of claim 3, wherein the computer is further programmed to generate the interpolated projection data using the second scan at an angle that is complementary to the given angle theta.

5. The CT system of claim 4, wherein the voxel is neither illuminated in one scan at a given angle nor a consecutive scan at the angle that is complementary to the given angle theta.

6. The CT system of claim 5, wherein the computer is further programmed to re-bin the acquired CT projection data from a fan geometry to a parallel geometry to generate re-binned projection data.

7. The CT system of claim 6, wherein the computer is further programmed to filter the re-binned projection data by a ramp filtering kernel.

8. A method of imaging, comprising:
passing an object through an opening of a rotatable gantry to be scanned;
receiving x-rays that pass through the object, in a pixelated detector positioned on the gantry, from which CT projection data is generated;
acquiring step-and-shoot (SAS) full scan CT projection data for a first scan and for a first rotation, and for a second scan and for a second rotation, wherein the first scan is axially offset from the second scan;
interpolating across the first and second scans to generate interpolated projection data; and
reconstructing an image based on the interpolated projection data.

9. The method of claim 8, further comprising:
identifying a voxel that is neither illuminated in one scan at a given angle nor a consecutive scan at an angle that is complementary to the given angle; and
generating the interpolated projection data based on the identified voxel.

10. The method of claim 9, further comprising generating the interpolated projection data using the second scan and based on the given angle theta.

11. The method of claim 10, further comprising generating the interpolated projection data using the second scan at the angle that is complementary to the given angle theta.

12. The method of claim 11, further comprising re-binning the acquired CT projection data from a fan geometry to a parallel geometry to generate re-binned projection data.

13. The method of claim 12, further comprising filtering the re-binned projection data by a ramp filtering kernel.

14. A computer readable storage medium having stored thereon a computer comprising instructions, which, when executed by a computer, cause the computer to:
acquire step-and-shoot (SAS) full scan CT projection data for a first scan and for a first rotation of a gantry, and for a second scan and for a second rotation of the gantry, wherein the first scan is axially offset from the second scan;
interpolate across the first and second scans to generate interpolated projection data; and
reconstruct an image based on the interpolated projection data.

15. The computer readable storage medium of claim 14, wherein the computer is further caused to:
identify a voxel that is neither illuminated in one scan at a given angle nor a consecutive scan at an angle that is complementary to the given angle; and
generate the interpolated projection data based on the identified voxel.

16. The computer readable storage medium of claim 15, wherein the computer is further caused to generate the interpolated projection data using the second scan and based on the given angle theta.

17. The computer readable storage medium of claim 16, wherein the computer is further caused to generate the interpolated projection data using the second scan at an angle that is complementary to the given angle theta.

18. The computer readable storage medium of claim 14, wherein the voxel is not illuminated in the second scan and at the angle that is complementary to the given angle theta.

19. The computer readable storage medium of claim 18, wherein the computer is further caused to re-bin the acquired CT projection data from a fan geometry to a parallel geometry to generate re-binned projection data.

20. The computer-readable storage medium of claim 19, wherein the computer is further caused to filter the re-binned projection data by a ramp filtering kernel.
